(19) United States
(12) Patent Application Publication
(10) Pub. No.: US 2019/0217865 A1
(43) Pub. Date: Jul. 18, 2019

(54) METHOD AND SYSTEM FOR DRUNK DRIVING PREVENTION

(71) Applicant: KHN Solutions, Inc., San Francisco, CA (US)

(72) Inventors: Keith Harry Nothacker, San Francisco, CA (US); Pauline Anne Basaran, Corte Madera, CA (US); Stacey Ilene Rettus, Greenbrae, CA (US); Zachary Michael Saul, San Francisco, CA (US)

(21) Appl. No.: 16/362,289

(22) Filed: Mar. 22, 2019

Related U.S. Application Data
(63) Continuation of application No. 15/205,876, filed on Jul. 8, 2016.
Application No. 15/205,876 is a continuation-in-part of application No. 14/973,227, filed on Dec. 17, 2015, now Pat. No. 9,915,644.
Application No. 14/973,227 is a continuation of application No. 14/602,919, filed on Jan. 22, 2015, now Pat. No. 9,250,228.
(60) Provisional application No. 62/219,306, filed on Sep. 16, 2015.
(60) Provisional application No. 62/189,871, filed on Jul. 8, 2015.
(60) Provisional application No. 61/930,062, filed on Jan. 22, 2014.

(51) Int. Cl. B60W 40/08; B60W 50/12; G01N 33/497; A61B 5/00; H04N 21/214; B60K 28/06; A61B 5/08

(52) U.S. Cl. CPC B60W 40/08; B60W 50/12; G01N 33/4972; A61B 5/4845; G06K 9/00892; B60K 28/06; A61B 5/082; B60W 2040/0836; H04N 21/2146

(57) ABSTRACT

A method and system for remotely monitoring intoxication of a user, including: detecting a vehicle-user interaction; prompting the user to provide a breath sample, in response to detecting the vehicle-user interaction; transmitting a breath sample signal, derived from reception of the breath sample from the user, to a mobile computing device; determining a value of an intoxication metric for the user based upon the breath sample signal; and based upon the value of the intoxication metric, performing an action to deter operation of the vehicle by the user.

### CROSS-REFERENCE TO RELATED APPLICATIONS

This application is a continuation of U.S. application Ser. No. 15/205,876 filed 8 Jul. 2016, which is a continuation-in-part application of U.S. application Ser. No. 14/973,227 filed 17 Dec. 2015, which is a continuation of U.S. application Ser. No. 14/602,919 filed 22 Jan. 2015, which claims the benefit of U.S. Provisional Application Ser. No. 61/930,062, filed 22 Jan. 2014, all of which are incorporated in their entireties herein by this reference. This application also claims the benefit of U.S. Provisional Application Ser. No. 62/219,306, filed 16 Sep. 2015, and U.S. Provisional Application Ser. No. 62/189,871, filed 8 Jul. 2015, which are each incorporated in their entireties herein by this reference.

### TECHNICAL FIELD

This invention relates generally to the intoxication monitoring device field, and more specifically to a new and useful method and system for drunk driving prevention.

### BACKGROUND

It is often desirable to analyze a biological sample from a person to detect substances carried in the biological sample. As such, breathalyzer devices are used to test the content of alcohol (i.e., ethanol) carried in an individual's breath, in order to determine a measure of alcohol consumed by the individual. The measure is typically presented as a blood alcohol content (BAC), which can provide an indication of a user's mental and/or physical adeptness resulting from intoxication. As such, BAC measures are also used to provide a basis for limits of alcohol consumption in relation to the performance of tasks, including driving a vehicle, operating machinery, and performing various tasks in a working environment. While current blood alcohol measuring devices are able to determine an individual's BAC, and are typically used in law enforcement settings, existing systems and methods configured to prevent drunk driving and/or any other activities that are dangerous to perform in an inebriated state are significantly deficient in many ways.

There is thus a need in the intoxication monitoring device field to create a new and useful method and system for drunk driving prevention. This invention provides such a new and useful method and system.

### BRIEF DESCRIPTION OF THE FIGURES

FIG. 1 depicts a flow chart of an embodiment of a method for drunk driving prevention;

FIG. 2 depicts an example of prompting an individual to provide a breath sample in an embodiment of a method for drunk driving prevention;

FIGS. 3A-3B depict examples of notifications in an embodiment of a method for drunk driving prevention;

FIGS. 4A-4J depict a specific application of at least a portion of an embodiment of a method for drunk driving prevention;

FIG. 5 depicts an embodiment of a system for drunk driving prevention;

FIG. 6 depicts an illustration of specific applications of at least a portion of an embodiment of a method for drunk driving prevention; and

FIG. 7 depicts a flow chart of an embodiment of a method for drunk driving prevention.

FIG. 8 depicts a flow chart of an embodiment of a method for drunk driving prevention.

FIG. 9 is a schematic representation of the method.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Method

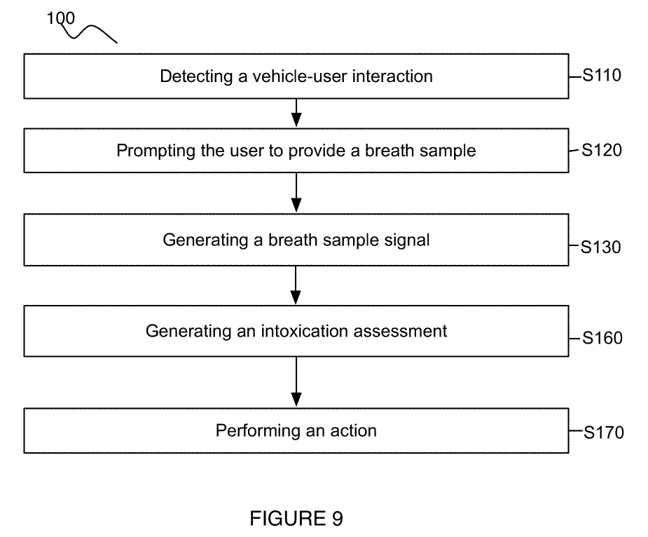

As shown in FIG. 9, the method 100 includes: detecting a vehicle-user interaction S110, prompting the user to provide a breath sample to provide a breath sample in response to vehicle-user interaction detection S120, generating a breath sample signal S130, generating an intoxication assessment of the user S160, and performing an action configured to deter drunk driving by the individual S170.

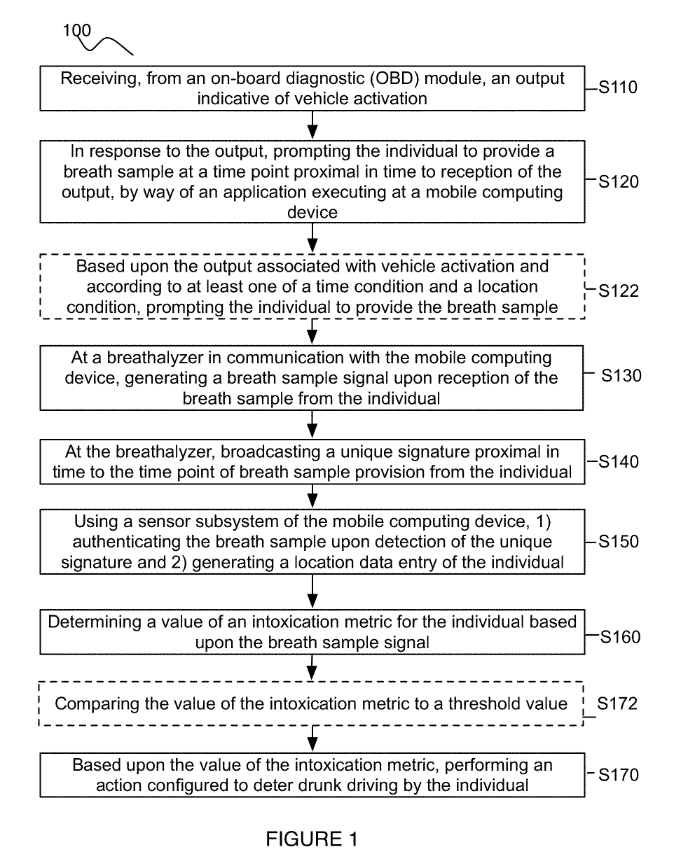

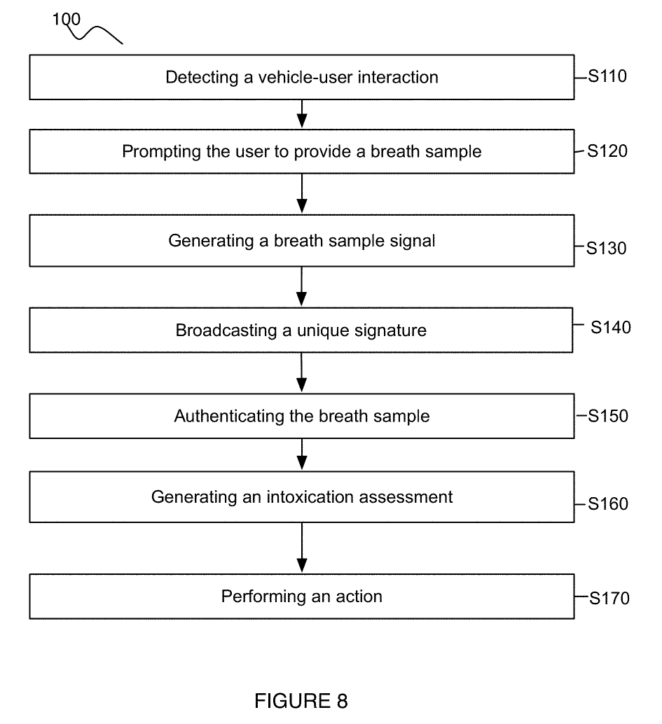

As shown in FIGS. 1, 4, 8, and 9, an embodiment of a method 100 for preventing drunk driving by an individual or user includes: detecting a vehicle-user interaction S110; prompting the user to provide a breath sample in response to detecting the vehicle-user interaction, through an application executing at a mobile computing device S120; at a breathalyzer in communication with the mobile computing device, generating a breath sample signal upon receipt of the breath sample from the individual S130, and broadcasting a unique signature within a predetermined time duration from breath sample receipt S140; authenticating the breath sample upon detection of the unique signature S150; generating an intoxication assessment for the individual based upon the breath sample signal S160; and based upon the value of the intoxication metric, performing a drunk driving deterrence action for the individual S170.

The method 100 functions to deter drunk driving behavior in individuals who are remote from an overseeing entity (e.g., parent, guardian, law enforcement officer, parole officer, relative, acquaintance, employment-related entity, etc.), in order to promote safe behaviors by an individual who is potentially in an inebriated state. In a specific application, the method 100 can allow a parent to monitor or otherwise be notified when the parent's child is about to begin driving after being in an alcohol use or substance use environment. In a related application, the method 100 can detect a state of inebriation of an individual, and automatically prevent the individual from driving a vehicle if he/or she is in an inebriated state. However, applications of the method 100 can be configured to prevent drunk driving by any other suitable individual, in any other suitable manner. Variations of the method 100 can further be adapted to prevent performance of any other suitable activity (e.g., operation of heavy machinery, etc.) by an individual when the individual is in an inebriated state.

The method 100 is preferably implemented by at least a portion of the system 200 described in Section 2 below. Furthermore, variations of the method 100 can be implemented at least in part by one or more embodiments, variations, and examples of system elements described in U.S. application Ser. No. 14/169,029 entitled “Method and System for Monitoring Intoxication” and filed on 30 Jan. 2014, U.S. application Ser. No. 14/602,909 entitled “Method and System for Remotely Monitoring Intoxication” and filed on 22 Jan. 2015, and U.S. application Ser. No. 14/631,125 entitled “Method and System for Monitoring Intoxication” and filed on 25 Feb. 2015, each of which is incorporated herein in its entirety by this reference. Variations of the method 100 can, however, be implemented at least in part using any other suitable system elements.

Block S110 recites: detecting a vehicle-user interaction, which functions to enable detection of a time point at which the individual, who may be inebriated, has activated a vehicle. Detecting a vehicle-user interaction preferably includes receiving an output indicative of a vehicle-user interaction from a diagnostic module electrically coupled to the vehicle, but can additionally or alternatively include detecting the vehicle-user interaction in any other suitable manner.

The vehicle-user interaction is preferably detected by a computing system in communication with the diagnostic module, but can be detected by any other suitable system.

The computing system can be a remote computing system (e.g., remote from the vehicle; a remote server, a cloud-based computing system, etc.), a mobile computing device (user device; e.g., smartphone, tablet, wearable, etc.), vehicle computing system (e.g., the central vehicle computer), a personal computer, a computing module of any other suitable computing device (e.g., mobile computing device, wearable computing device, etc.), or be any other suitable computing system. When the computing system is the remote computing system, the computing system can receive the output: directly from the diagnostic module (e.g., through a shared wireless communication channel), indirectly from the diagnostic module (e.g., through an intermediary user device proximal the vehicle or diagnostic module, through an intermediary remote computing system associated with the diagnostic module, etc.), or through any other suitable data path. The user device can include inputs (e.g., touchscreen, camera, microphone, etc.), outputs (e.g., display, haptic system, speaker, etc.), sensors (e.g., accelerometers, gyroscopes, temperature sensors, light sensors, audio sensors, etc.), on-board power (e.g., battery), communication systems (radios, e.g., BLE, NFC, WiFi, Bluetooth 4.0, RF, cellular, etc.), location systems.

The diagnostic module can be an on-board diagnostic (OBD) module, a diagnostic module integrated into the vehicle (e.g., vehicle sensor system, vehicle communication system, etc.), a camera system on-board the vehicle, or be any other suitable system.

In a first variation, Block S110 is preferably implemented at a computing system in communication with the on-board diagnostic (OBD) module of a vehicle associated with the individual, wherein the OBD module includes hardware that generates data associated with statuses of subsystems of the vehicle. In variations, the OBD module can have any of the following interfaces with the vehicle: an assembly line diagnostic link (ALDL) interface, an OBD-1 interface, an OBD-1.5 interface, an OBD-II interface, a European OBD interface, a Japanese OBD interface, an ADR79 interface, and any other suitable interface that allows access and detection of vehicle subsystem statuses. In specific examples, the OBD modules can comprise one of: an Automatic OBD adaptor (i.e., from Automatic Labs) and a Metromile OBD adaptor (i.e., from Metromile, Inc.) that plugs into an OBD port of the vehicle and communicates with a mobile computing device of the computing system (e.g., by way of a Bluetooth connection, by way of any other wireless connection, by way of a wired connection, etc.) in transmitting an output associated with a vehicle-user interaction to the computing system.

In a second variation, the diagnostic module can be an integrated subsystem of the factory-produced vehicle (e.g., an original equipment manufacturer (OEM) module integrated into the vehicle computer) instead of a removable adaptor. In a third variation, the diagnostic module can be configured to receive input and control vehicle parameters. In specific examples of related variations, the diagnostic module (as a removable adaptor or as an integrated module) can receive instructions over a wireless data link (e.g., Bluetooth 4.0, near-field-communications (NFC), in-vehicle WiFi, etc.) or wired data link (e.g., universal serial bus (USB), Ethernet, etc.) and implement the instructions in order to limit vehicle parameters (e.g., the maximum speed of the vehicle) to a predetermined value and/or range of values (e.g., 25 mph, 25-30 mph, etc.) in cooperation with the vehicle computer and/or additional vehicle hardware and/or software.

The output indicative of a vehicle-user interaction in variations of Block S110 is preferably generated by the OBD module upon accessing (by the user or an individual spatiotemporally associated with the user) an ignition subsystem of the vehicle and detecting when the ignition subsystem of the vehicle is activated. However, the output of Block S110 can be any other suitable output that indicates that the vehicle is entering an in-use status (e.g., based upon detection of unlocking of the vehicle, based upon opening of the trunk of the vehicle, based upon activation of any suitable electrical subsystem of the vehicle, based upon activation of any suitable mechanical subsystem of the vehicle, based upon activation of any suitable electromechanical subsystem of the vehicle, etc.), be any suitable output that indicates that the user is proximal the vehicle (e.g., based on user device connection to the diagnostic module or vehicle, diagnostic module accelerometer measurements indicative of door opening and/or closing, diagnostic module microphone measurements indicative of user presence, etc.), or be any other suitable output. Furthermore, the output can be transmitted (e.g., broadcast, sent to a specific endpoint, etc.) as long as the associated subsystem is in a triggering state (e.g., activated state), and/or can be transmitted in response to changes in state of a vehicle subsystem. In specific examples, the OBD module can broadcast a signal indicating that the vehicle is in an activated state (e.g., the electrical subsystems are activated, the engine is running, etc.) for the duration that the vehicle is in operation, and this signal (i.e., the output) can be received at any time point or time points during which it is broadcast. In other specific examples, the OBD module can broadcast a signal at a time point within a short time interval (e.g., 5 seconds, 10 seconds) of vehicle ignition, at which time point the signal is received by the mobile computing device of the user over a wireless data transfer link.

Furthermore, data accessed using the OBD module associated with the method 100 can be used to augment or otherwise replace authentication and/or drunk driving deterrence actions in subsequent blocks of the method 100. For instance, in interfacing with sensor systems of the vehicle, the OBD module can provide one or more of: body weight information associated with one or more seat positions in the vehicle, body temperature information associated with one or more seat positions in the vehicle, biometric data (e.g., heart rate data, galvanic skin response, motion data, body-position data, posture data, ergonomics-related data, mirror position data, etc.) associated with the driver of the vehicle (e.g., from sensors integrated into vehicle system control input devices proximal the driver's seat), authentication data (e.g., voice recognition data, facial recognition data, fingerprint recognition data, etc.) associated with the driver and/or passengers of the vehicle, and any other suitable information. As such, information from the OBD module can be used to identify the driver and/or passengers of the vehicle (e.g., based upon processing of voice data, based upon processing of image data, based upon processing of video data, based upon processing of fingerprint data, based upon processing of weight data, based upon processing of body temperature data, based upon processing of biometric data, etc.). Additionally or alternatively, this and/or other information from the OBD module can be used to determine impairment of the individual intending to drive the vehicle.

In implementations of Block S110, the computing system may have an associated log (e.g., a log stored in memory) of multiple identified diagnostic modules associated with the individual. For instance, for an individual who has access to a set of vehicles, identifiers of diagnostic modules (e.g., OBD modules, vehicles) of each of the set of vehicles can be pre-recorded and stored in the log. Then, upon detection that one of the vehicles associated with the identified diagnostic modules is in an activated state, Block S110 can include providing an output indicative of a vehicle-user interaction to the computing system for triggering subsequent blocks of the method 100. Additionally or alternatively, each diagnostic module can be associated with a set of user identifiers (e.g., in an on-board or remote log), wherein the output is provided in response to diagnostic module detection of the user associated with the user identifier (e.g., through the user's device, etc.). However, multiple diagnostic modules and/or user identifiers can be previously unassociated or otherwise associated.

In a specific example of Block S110, a parent who has given his/her child access to the family vehicle can install an OBD adaptor in the vehicle, wherein the OBD adaptor detects when the vehicle is in an activated state and transmits signals associated with activation to the computing system, by way of a mobile computing device (e.g., smartphone, tablet, etc.) in communication with the OBD adaptor. In more detail, whenever the child activates the vehicle, the OBD adaptor can be configured to send a “vehicle activation” signal from the OBD module to the mobile computing device and from the mobile computing device to the cloud (e.g., by way of a native application executing on the mobile computing device). The signal or derivatives thereof can then be transmitted to a server by way of a software developer's kit (SDK) of the OBD adaptor. However, variations of Block S110 associated with detection of a vehicle-user interaction can alternatively be implemented in any other suitable manner. In a second specific example, the diagnostic module sends a “vehicle activation” signal to a first server (associated with an OBD entity) in response to vehicle activation and the first server sends a vehicle activation notification to a second server (associated with the breathalyzer), wherein the second server can trigger the breathalyzation test for one or more users in response to vehicle activation notification.

In some variations, detecting a vehicle-user interaction in Block S110 can include detecting, collecting, analyzing, and/or evaluating environmental data pertaining to the user. The environmental data can be used to detect the vehicle-user interaction, be used to identify a user to be tested, or be used in any other suitable manner. Environmental data can be location data, temporal data, image data, audio data, or any other suitable data indicative that the user has interacted with, is interacting with, is about to interact with, or intends to interact with a vehicle. Environmental data is preferably collected and/or generated by the computing system, and more preferably by the mobile computing device of the user, but can alternatively be generated by the vehicle (e.g., a sensor of the vehicle), a mobile device of an individual other than the user, another portion of the computing system, or any other suitable element. Detecting environmental data preferably occurs contemporaneously with detecting a vehicle-user interaction (e.g., in real- or near-real time; within a time threshold, such as 5 s; etc.), but can alternatively occur prior to the vehicle-user interaction, subsequent to the vehicle-user interaction, or at any other suitable time point.

In a first variation, the mobile computing device can detect the environmental data using a sensor and/or sensor(s) of the mobile device. For example, a GPS location sensor of the mobile device can detect that user Tim is moving at a rate of speed consistent with vehicle travel (e.g., greater than 15 mph), wherein this data can be interpreted as indicative of a vehicle-user interaction in real-, near-real, or any other suitable time from sensor signal recordation. In another example, a microphone of the user's mobile computing device can detect an audio signature of a car door opening and/or unlocking, or a voice recognition module of the mobile computing device can detect speech patterns indicative of vehicle operation (e.g., user Tim says, “Turn on the car!”).

In a second variation, the computing system can detect and synthesize social networking data associated with the user to determine that the user intends to operate a vehicle. In relation to this and other examples and variations, social networking data (social media data, social networking system (SNS) data) can include any form of data (e.g., images, videos, text messages, audio messages, etc.) made publically, semi-publically, or privately available on a network accessible by one or more individuals associated with the user (including the user him or herself), and/or by applications granted access to the social media data of the user. For example, user Tim posts a series of text based messages to his social media account indicating he intends to drive and that he has consumed alcohol, which is detected by a client application running on Tim's cell phone and/or a remote computing system, and transmitted to a cloud-based application which in turn determines that Tim intends to attempt to drive while inebriated, based on a keyword analysis of Tim's social media post. Additionally or alternatively, in this and other variations, environmental data can be included in detecting a vehicle-user interaction in any other suitable manner, and/or included in other Blocks of the method 100. In relation to this example and in related examples of these variation of Block S110, detecting a vehicle-user interaction can include multiple sub-blocks involving data collection, transmission, receipt, and analysis in any suitable order.

In alternative variations of the method 100 adapted to prevent performance of any other activity (e.g., operation of heavy machinery, etc.) by an individual when the individual is in an inebriated state, Block S110 can include setting a trigger associated with detection that the individual is about to initiate the activity. The trigger can be detected from the environmental data, detected from auxiliary systems (e.g., monitoring systems), or otherwise determined. In examples, the trigger can be associated with one or more of: a location of the individual (e.g., by using GPS or other location determining system elements), a physiological state of the user (e.g., by using a biometric monitoring system associated with the individual), apparatus associated with the activity of the individual (e.g., by detecting activation of heavy machinery, by detecting access or activation of a computing module of a computing module used by the individual to perform the activity), and any other suitable trigger.

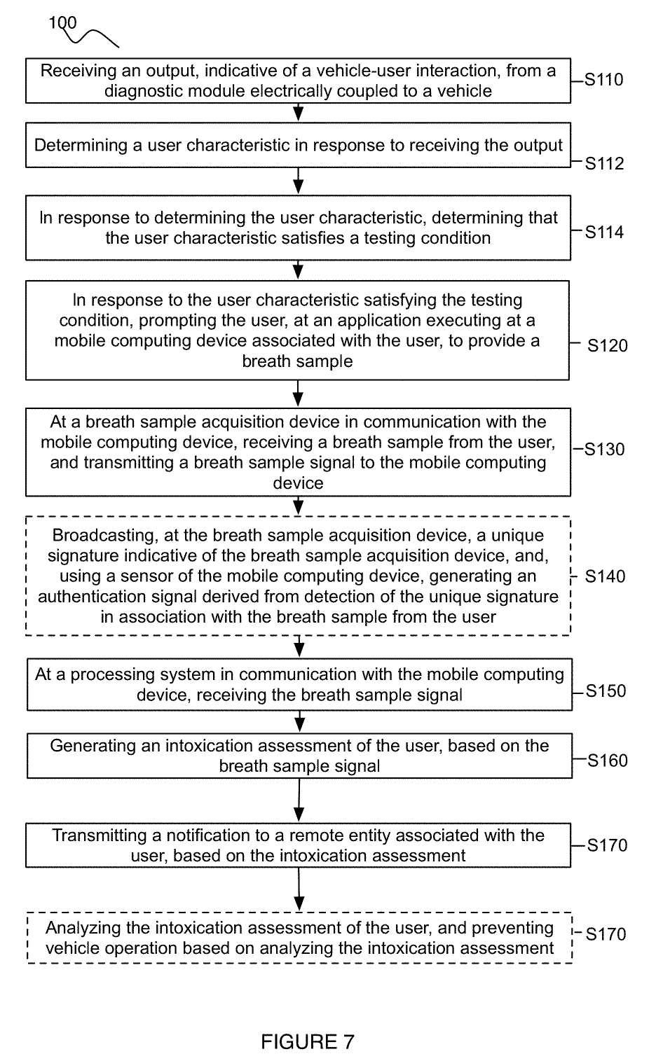

As shown in FIG. 7. S112 can include determining the user characteristic, which function to obtain information about the user that can be used to assess whether the user should be prompted to provide a breath sample.

The user characteristics can be determined after receiving the output from the diagnostic module indicative of a vehicle-user interaction, but can alternatively be determined prior to receiving the output, substantially simultaneously to receiving the output and/or detecting the vehicle-user interaction, or in variations in which no diagnostic module output is received. The user characteristic is preferably determined by the computing system, which can include the mobile computing device and associated sensors, the vehicle computing system, the diagnostic module, and/or any other suitable computing modules. Alternatively or additionally, the user characteristic can be determined by another individual besides the user, by a combination of the computing system and manually-and/or automatically-generated inputs, or in any other suitable manner. Determining the user characteristic can include, in variations, detecting, measuring, receiving, retrieving, evaluating, generating, and/or recording the user characteristic. The user characteristic is preferably data representative of an aspect of the user that can be used to uniquely identify the user, but can alternatively be data associated with the user that can be used to algorithmically determine that the user should be prompted to provide a breath sample. The user characteristic can additionally or alternatively be biometric data of the user, user identity data, environmental data associated with the user (e.g., user location, the time interval in which the user is interacting with the vehicle, etc.), or any other suitable characteristic of the user.

User biometric data (biometrics of the user) used in determining a user characteristic can include the user's height, weight, fingerprint, iris pattern, voice pattern, heart rate, temperature, pupil dilation, or any other suitable biologically-derived parameter associated with the user. The biometric data is preferably collected, generated, and/or transmitted by the computing system, such as the mobile computing device, the diagnostic module, a sensor associated with the vehicle, or any other suitable module for obtaining biometric data. The biometric data can be used to identify the user (e.g., by fingerprint analysis), and/or to indicate a likelihood of inebriation of the user (e.g., by analyzing pupil dilation in conjunction with ambient light levels) for use in subsequent Blocks of the method, such as determining that the user characteristic satisfies a testing condition S114. The biometric data can also be used in any other suitable manner.

User identity data can include a user name, address, driver's license number, user account information (e.g., user account identifier), mobile device identifier, social networking system account, or any other suitable data identifying the user.

In a first variation of Block S112, determining a user characteristic includes detecting a unique identifier of the mobile computing device associated with the user. In this variation, the user is preferably sufficiently uniquely associated with the mobile device that a unique identifier of the mobile computing device (e.g., a serial number, a user account native to the mobile device, etc.) can be used to characterize the user him or herself (e.g., as a user characteristic). Alternatively, the unique identifier of the mobile device can be associated with the user by a user account, which the user logs into (e.g., via a native application running on the mobile device, a mobile web interface accessed via the mobile device, etc.) when using the mobile device. The unique identifier of the mobile device can additionally or alternatively be used to characterize the user in any other suitable manner. In an example of this variation, the user characteristic is the name of the user (e.g., Tim Beaver), which is associated with a user account stored on a remote server, and it is determined by a native application running on Tim's smartphone by retrieving Tim's name from a stored database (e.g., stored at the smartphone and accessible by the native application, stored remotely and accessible by the native application over a wireless data connection, etc.).

In a second variation of Block S112, the user characteristic can be determined based on collection and analysis of environmental data associated with the user. For example, determining a user characteristic can include analyzing a photo taken with a camera of the mobile computing device or vehicle that includes visual indications that the user is drinking (e.g., alcohol containers in the image, dilation of the user's pupils, barstools visible in the background of the image, etc.), and producing an analysis of the likelihood that the user has been drinking based on assessing these visual indications (e.g., using image analysis techniques, feature detection techniques, artificial intelligence techniques, such as neural networks, etc.). Additionally or alternatively, the user characteristic can be determined from any suitable combination of information and/or data characterizing the user (e.g., location of the user, time stamp data at the time point that a vehicle-user interaction is detected, social network data, user profile data, etc.) and/or combinations of such data (e.g., location in combination with time stamp data, social network data in combination with user profile data, etc.). However, any other suitable user information can be determined in any other suitable manner.

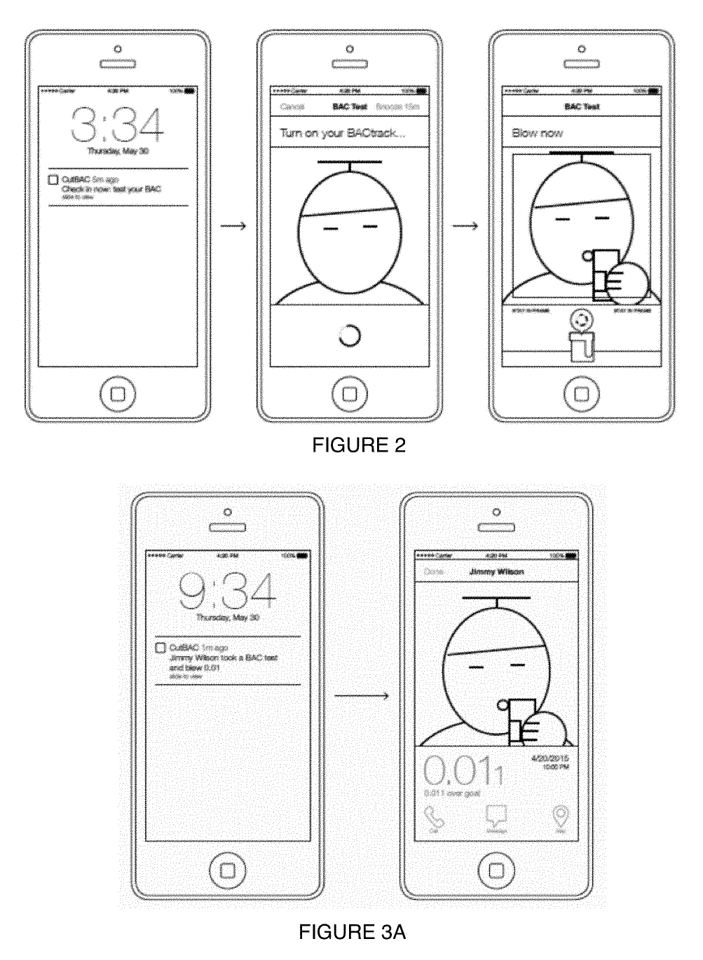

Block S120 recites: prompting the user to provide a breath sample at a time point proximal in time to vehicle-user interaction detection, which functions to prompt the individual to provide the breath sample prior to driving of the vehicle (or performing any other activity that is life-threatening in an inebriated state), in order to deter the individual from driving in an inebriated state. Block S120 is preferably implemented by way of an application executing at a mobile computing device in communication with the computing system, wherein the application guides the user (individual) in providing the breath sample, as in Block S130. However, Block S120 can additionally or alternatively be implemented using any other suitable computing device (or non-computing entity) that can be used to prompt the user to provide a breath sample. In Block S120, prompting is preferably performed using one or more of: display functions of a display of the mobile computing device (or breathalyzer), an example of which is shown in FIG. 2, speaker functions of an audio-output element of the mobile computing device (or breathalyzer), haptic functions of an actuation element (e.g., vibration motor) of the mobile computing device, visual signal functions of a light emitting element (e.g., LED) of the mobile computing device (or breathalyzer), and/or any other suitable function of the mobile computing device (or breathalyzer). Block S120 is preferably performed in response to the user characteristic satisfying the testing condition S114, but can alternatively be performed in response to receiving the output of the diagnostic module, or in response to and/or based on any other suitable input or condition. Block S120 is preferably performed at a second time point, wherein the vehicle-user interaction is determined at a first time point. The first time point can be different from (e.g., before or after) the second time point, or be the same as the second time point.

The individual can be prompted to provide the breath sample S120 by a client on a mobile computing device (e.g., smartphone, wearable computing system, laptop, etc.) associated with the user, but can alternatively be prompted by a client running on the vehicle (e.g., through a vehicle display), by the breath sample acquisition device, or through any other suitable component. In one example, a native application executing at the mobile computing device can provide a graphic and/or textual message, using the display of the mobile computing device, to cue the user to provide a breath sample upon pairing of the mobile computing device with an associated breath sample acquisition device. In a specific example, the native application can operate the display of the mobile computing device to display a graphical representation of the breath sample acquisition device that has been paired with the mobile computing device, along with textual messages that indicate an initiation time point of breath sample provision, an appropriate duration of breath sample provision, and/or any other suitable set of instructions or data in order to achieve a suitable breath sample. Additionally or alternatively, prompting in Block S120 can be performed using the breathalyzer described in Block S130, for instance, using LED signals to prompt sample provision. Additionally or alternatively, prompting in Block S120 can be performed in a non-electronic format, for instance, by way of an interaction (e.g., a verbal interaction, an interaction in writing, etc.) between the user and another human entity associated with the user.

In Block S120, prompting the individual to provide the breath sample can optionally include Block S114: determining whether the individual satisfies a testing condition. Block S114 functions to assess whether the user should be prompted for a breath sample or otherwise have their inebriation status tested when the user interacts with a vehicle, preferably incorporating the determined user characteristic into the assessment. Block 114 can be performed using the user characteristics determined in Block S112, the environmental data determined in Block S110, or using any other suitable data.

Block S114 is preferably performed by the computing system in communication with the diagnostic module, which can include the mobile computing device, and/or a remote server, but can alternatively be performed by any other suitable portion of the computing system.

In a first variation, Block S114 includes determining whether a time-and/or location-based condition has been satisfied. The location- and/or time-based condition can be associated with states in which the individual has a higher probability of substance use or alcohol consumption, or be any other suitable time- and/or location-based condition. The user is preferably tested (e.g., prompted to provide the breath sample) when the time and/or location condition is met, but any other suitable action can be alternatively or additionally performed. Thus, Block S114 can include Block S122, which recites: based upon the detection of a vehicle-user interaction and according to at least one of a time condition and a location condition, prompting the individual to provide the breath sample. In variations, the time condition can be a time condition associated with times (e.g., weekend time points, evening time points, etc.) at which the individual is more likely to have consumed alcohol. In other variations, the time condition can be a time window after vehicle-user interaction that precedes vehicle operation. In still other variations, the time condition can be a time window after vehicle operation (e.g., to monitor a known driver or passenger) and/or a predetermined frequency after vehicle operation. This can function to automatically test a user that has previously driven or ridden in a vehicle (e.g., to a bar or party). Additionally or alternatively, in variations, the location condition can be a location condition associated with locations (e.g., bars, lounges, restaurants, clubs, homes of friends of the individual, liquor stores, etc.) at which the individual is more likely to have consumed alcohol. The condition(s) of Block S122 can be set by an entity (e.g., acquaintance, employment-related entity, relative, friend, parole officer, etc.) monitoring the individual, based upon inputs provided by the entity at an input device in communication with the computing system. The conditions can additionally or alternatively be automatically generated based upon behavioral and/or physiological analyses of the individual and/or populations of individuals related to times and/or locations associated with a higher probability of intoxication. For example, the time condition can be determined based on the user's alcohol metabolization rate, the user's recent intoxication metric values, the estimated amount of alcohol consumed by the user, or otherwise determined. The conditions can additionally or alternatively be automatically determined based on social networking data automatically and/or manually analyzed by the computing system and/or entities associated with a higher probability of intoxication (e.g., when a party is detected in the user's social networking schedule).

In a first example of the first variation of S114, satisfaction of a time condition can be determined by comparing a time at which the vehicle transitions from an inactive to an active state (as facilitated by Block S110), to the time condition (e.g., Friday after 10 PM), and triggering prompting if the time condition is satisfied. In a second example, satisfaction of a location condition can be determined by identifying a location that the individual has visited prior to activation of the vehicle (e.g., using a GPS subsystem, using any other suitable location determining technology), categorizing the location of the individual, and comparing the location of the individual to parameters of the location condition. In a third example, satisfaction of a location condition can be determined by identifying a location that the individual has visited after vehicle operation (e.g., using a GPS subsystem, using any other suitable location determining technology), categorizing the location of the individual, and comparing the location of the individual to parameters of the location condition. In a specific example, a remote server may receive an output indicating that user Tim drove a vehicle within the last hour, and transmit instructions to Tim's mobile device to prompt Tim to provide a breath sample for recordation and/or data logging purposes periodically, until the intoxication metric value falls below the threshold level.

In a second variation, Block 114 includes determining whether the user identifier is included within a set of users to be breathalyzed (monitored user set). The user is preferably tested (e.g., prompted to provide the breath sample) when the user is within the monitored user set, but any other suitable action can be alternatively or additionally performed. Thus, Block S114 can include: based upon the detection of a vehicle-user interaction and in response to the user identifier matching a user identifier within the monitored user set, prompting the individual to provide the breath sample. The monitored user set can include identifiers for users to be tested, or include identifiers for any other suitable user. The monitored user set can be a list of user identifiers (e.g., user names), a set of pointers to user profiles, or have any other suitable data structure. Each user and/or user identifier can be associated with a different user profile, but can alternatively share a profile. Each user profile can include user preferences, permissions, testing conditions (e.g., subscription status, monitoring status, etc.), or include any other suitable information. The user identifiers included within the monitored user set can include user identifiers associated with a specified subscription status (e.g., a paid status; an unpaid status; a registered status; etc.), user identifiers associated with a specified monitoring status (e.g., “monitoring on”), user identifiers for past drivers or passengers of a specific vehicle, or any other suitable user identifier. User identifiers can be included within the monitored user set by the user themselves (e.g., through the user client), by a second user (e.g., a parent, a probation officer, etc.), automatically included (e.g., in response to subscription payment by the user or secondary user), or be otherwise included. The monitored user set can be universal across all vehicles (e.g., referenced for any vehicle), specific to a vehicle set (e.g., only referenced when a vehicle within the set is being operated), specific to a single vehicle, associated with any other suitable set of vehicles, or unassociated with any vehicles.

In one example of the second variation of S114, the user can be prompted to provide a breath sample in response to determination that the user identifier is included within the set. In this example, the user can optionally not be prompted to provide a breath sample in response to determination that the user identifier is not included within the set. However, user identifier inclusion within the set can be otherwise used. This example implementation may arise if a vehicle has multiple possible users, of which some subset are required to provide a breath sample upon interacting with the vehicle.

In a third variation, Block 114 includes determining whether the user is the driver of the vehicle. The user is preferably tested (e.g., prompted to provide the breath sample) when the user is likely the vehicle driver (e.g., actual or intended), but any other suitable action can be alternatively or additionally performed. This can function to differentiate between drivers and passengers, and prevent false positive events (e.g., prevent cases where a drunk passenger precludes vehicle operation because they failed the breathalyzation test). In other use cases, such as utilizing remote blood alcohol testing to provide reward incentives, the user may be rewarded for providing a breath sample that indicates that the user is inebriated when it has been determined that the user is not the driver of the vehicle. Thus, Block S114 can include: based upon the detection of a vehicle-user interaction and in response to the determination that the user is likely the vehicle driver, prompting the user to provide the breath sample. In some variants, ensuring that the tested user is the vehicle driver can be achieved by sending the prompt through a short-range communication protocol from the diagnostic module.

In a first embodiment of the third variation of Block S114, determining whether the user is the driver can include: determining user proximity to the driver volume. User proximity to the driver volume can be determined when the user is proximal: the driver seat, the steering column, the OBD port, the pedals, the driver door, or proximal any other suitable driver volume component located within or otherwise associated with the driver volume. User proximity to the driver volume can be determined through: user device connection to the reference component through a short-range communication protocol (e.g., Bluetooth, BLE, NFC, etc.), detection of a user characteristic indicative of the user within the driver volume, or otherwise determined.

In a first variant of the first embodiment, determining user proximity to the driver volume can include: determining user proximity to the diagnostic module (e.g., wherein the diagnostic module is an OBD module plugged into the OBD port). In a first example, user proximity to the diagnostic module can be determined through pairing of the user's mobile device and the diagnostic module over a short range wireless data transfer link (e.g., Bluetooth, NFC, or other range-limited wireless communication protocols), wherein the diagnostic module is coupled to the vehicle in a location proximal the steering wheel of the vehicle (e.g., within one foot, within the projected enclosed volume defined by the furthest extents of the driver's seat and the steering wheel, in the vicinity of the dash board on the side of the vehicle on which the steering vehicle is located, etc.). In a specific example, user Tim's mobile phone may pair with an OBD module connected to his vehicle, detect an output that indicates the vehicle has been turned on, and immediately prompt Tim to provide a breath sample. Thus, in this example, the fact that the user is the probable driver (i.e., the user characteristic in this example) is inferred from the user's proximity to the driver's seat and/or steering wheel, as determined from the pairing of the user's smartphone (e.g., in the user's pants pocket) with the diagnostic module (e.g., a Bluetooth-enabled OBD module) over a wireless link of limited, predetermined physical range.

In a second variant of the first embodiment, determining user proximity to the driver volume can include: receiving data indicative of driver identification at the native application running on the user device. The data itself can indicate whether user is the driver (e.g., the native application prompts the user to answer “yes” or “no” to the prompted question, “Are you the driver of the vehicle?”), or indicate any other suitable information.

In a third variant of the first embodiment, determining user proximity to the driver volume can include: detecting biometric values associated with the user from biometric sensor measurements determined by a driver volume component. For example, the user weight can be detected by a sensor of the driver's seat of the vehicle and communicated to the computing system, wherein the computing system compares the measured weight with a known weight (and/or weight range) of the user (e.g., associated with the user by way of a user profile and/or user account) in order to analyze whether the user is sitting in the driver's seat of the vehicle. The population of users that are considered in this analysis can be limited to users that have historically driven the vehicle, users connected to past drivers within a given connection degree (e.g., based on social network analysis), be unlimited (e.g., all users), or be any other suitable set of users. In a specific example, Tim can be prompted to provide a breath sample in response to the vehicle detecting Tim's weight in the driver seat.

Block S114 can additionally or alternatively include prompting the individual according to any other suitable condition derived from additional data pertaining to the individual. For instance, one or more of: accessing of calendars of the individual, accessing of online social network posts or location tags of the individual, accessing and processing communications (e.g., phone calls of the individual, text messages of the individual, emails of the individual, etc.), environmental data as described above or any other suitable environmental data, the user characteristic as determined in Block S112, and any other suitable data can be used to determine if the individual has a higher probability of being in an inebriated state and trigger prompting of the individual to provide a breath sample prior to driving.

However, the individual can be prompted to provide the breath sample in response to satisfaction of any other suitable condition, upon occurrence of any other suitable event, or at any other suitable time.

In any of the above variations of Block S120, prompting can be automatic, such that once the output indicative of a vehicle-user interaction is received and/or appropriate conditions are met, the user is automatically prompted to provide the breath sample. Additionally or alternatively, prompting can be semi-manual or manual, such that information associated with the individual upon detection of a vehicle-user interaction is provided to an overseeing entity (e.g., guardian, etc.), who decides whether or not the individual should provide a breath sample.

In one variation of automatic prompting, the remote computing system automatically generates a testing instruction upon determination that the testing conditions are met and transmits the instruction to the user device associated with the user to be tested, wherein the user device presents the testing prompt. The user device identifier, native application, account, or other endpoint identifier can be determined based on the determined user characteristic or otherwise determined.

In a second variation of automatic prompting, the remote computing system automatically generates a testing instruction upon determination that the testing conditions are met and transmits the instruction to the diagnostic module from which the output was received, wherein the diagnostic module transmits (e.g., broadcasts, sends, etc.) the instruction to connected and/or proximal user devices. The receiving user device(s) can present the prompt in response to instruction receipt. The testing instruction can be addressed (e.g., to the driver's user device) or unaddressed (e.g., wherein all scanning user devices receive the instruction). The testing instruction can be sent directly from the remote computing system to the diagnostic module, be sent through an intermediary remote computing system (e.g., a remote computing system connected to the diagnostic module), or otherwise sent to the diagnostic module.

In a third variation of automatic prompting, the user device automatically presents the prompt in response to user device determination that the testing conditions are met (e.g., wherein the user device stores the testing conditions to be met).

In a fourth variation of automatic prompting, the diagnostic module automatically generates and controls prompt presentation (e.g., on the user device or vehicle) in response to diagnostic module determination that the testing conditions are met (e.g., wherein the user device stores the testing conditions to be met).

Block S120 can, however, be implemented in any other suitable manner.

Block S130 recites: at a breathalyzer in communication with the mobile computing device, generating a breath sample signal upon reception of the breath sample from the individual. Block S130 can additionally or alternatively include transmitting a breath sample signal, derived from reception of the breath sample from the individual, to the mobile computing device implemented in embodiments of the method 100. Block S130 can additionally or alternatively include transmitting a breath sample signal to the mobile computing device. However, Block 130 can be otherwise performed

Block S130 functions to receive a sample from the user from which a signal can be generated and a value of an intoxication metric can be determined, in subsequent blocks of the method 100. Preferably, the breathalyzer and the mobile computing device are uniquely associated with each other and with the individual, in order to enable verification of the source of breath samples provided by the individual. Furthermore, the breathalyzer is preferably uncoupled from the vehicle and easily transported, such that the individual can keep the breathalyzer on himself/herself while performing various activities. However, the breathalyzer, the mobile computing device, and the vehicle can alternatively be configured in any other suitable manner.

In Block S130, the breath sample signal is preferably generated at a fuel cell sensor that enables measurement of the individual's blood alcohol content (BAC), and/or other intoxication factor indicative of sobriety, by an electrochemical process. In relation to the fuel cell sensor, generating the breath sample signal can include producing an electrical current in response to oxidation of alcohol carried in the breath sample provided by the user, wherein the magnitude of the produced electrical current varies in a predictable manner according to the amount (e.g., relative volume) of alcohol carried in the breath sample. In an alternative variation, generating the breath sample signal can be implemented at a semiconductor sensor that produces a change in electrical resistance in response to an alcohol-dioxide reaction, wherein the magnitude of the change in resistance varies in a predictable manner according to the amount (e.g., relative volume) of alcohol carried in the breath sample. In other variations however, Block S130 can additionally or alternatively include generating a breath sample signal at a spectrophotometer configured to produce a signal in response to absorbed or emitted light from alcohol molecules carried in the breath sample from the user. Generating the breath sample signal in Block S130 can, however, include generating a signal at any suitable element configured to respond to alcohol in a sample from the user.

Block S130 can additionally or alternatively include generating data for assessing impairment of the user in subsequent blocks of the method 100, without direct assessment of a BAC of the individual. For instance, Block S130 can additionally or alternatively include assessing impairment of the user based upon the user's performance of one or more tasks (e.g., tasks derived from a sobriety test), some embodiments, variations, and examples of which are described in U.S. application Ser. No. 14/169,029 entitled “Method and System for Monitoring Intoxication” and filed on 30 Jan. 2014, which is herein incorporated in its entirety by this reference. As such, BAC measurements in Block S130 can be supplemented with or otherwise replaced with alternative methods of assessing impairment of the user.

In a specific example of Block S130, receiving the breath sample can include receiving the breath sample at a cavity of a breathalyzer, wherein the cavity includes a first aperture and a second aperture configured to facilitate breath intake and outflow, respectively. In the specific example, the cavity is in communication with a fuel cell sensor that receives a metered volume of the breath sample, wherein the fuel cell sensor facilitates generation of a breath sample signal by an electrochemical process. Specifically, the fuel cell sensor produces an electrical current in response to oxidation of alcohol carried in the breath sample provided by the individual, wherein the magnitude of the produced electrical current varies in a predictable manner according to the amount (e.g., relative volume) of alcohol carried in the breath sample. As such, in the specific example, the breath sample signal comprises an electrical current magnitude that can be detected and processed to determine a value of an intoxication metric indicative of the sobriety of the individual. Variations of the specific example can, however, include generation of any other suitable type of electrical signal in response to a received breath sample from the individual.

In alternative variations of Block S130 of the method 100, receiving a breath sample from the individual can be substituted with or supplemented with receiving any one or more of: urine samples, blood samples, interstitial fluid samples, and any other suitable sample (e.g., from a transdermal sensor) that can be used to assess the user's substance use. Furthermore, in relation to any of the above types of samples, samples used to determine sobriety and substance usage by the individual are preferably received from the user in a non-invasive manner; however, samples can additionally or alternatively be received in a minimally invasive or invasive manner. Furthermore, in some variations, a signal generated in response to a received sample can be generated without directly collecting a sample from the individual. For example, a signal can be generated in an indirect manner, as derived from an interaction between a stimulus and the user's body (e.g., spectrometer-based analysis of light transmitted from a user's blood vessels).

While some embodiments, variations, and examples of Blocks S110-S130 are described above with respect to incorporation a mobile computing device, alternative variations of Blocks S110-S130 can omit involvement of or otherwise reduce reliance upon a mobile computing device in initiating breath sample provision based upon detection of a vehicle-user interaction. For instance, the OBD module associated with Block S110 can additionally or alternatively include a cellular module (or other wired/wireless data communication module), which can allow the method 100 to continue to be implemented (e.g., if there is a failure associated with the individual's mobile computing device). Additionally or alternatively, data transmitted using the data communication module integrated with the OBD module can be used for any other suitable purpose. Additionally or alternatively, direct communication between the OBD module and a breathalyzer associated with the method 100 can be used for initiation of breath sample provision, without involvement of an intermediary mobile computing device.

Block S140 recites: within a time interval, broadcasting a unique signature indicative of the breath sample acquisition device, wherein the time interval is proximal in time to the first time point, and Block S150 recites: generating an authentication signal derived from detection of the unique signature in association with the breath sample from the user using a sensor of the mobile computing device. Block S140 functions to provide a detectable signature, in association with breath sample provision by the individual, that can be used to validate the breath sample acquisition device as the source of the breath sample signal generated in Block S130, in order to authenticate the breath sample in Block S150. As such, Blocks S140 and S150 prevent fraudulent activity involving another entity (e.g., an entity who is not the individual) and/or another breath sample acquisition device. In Block S140, the mobile computing device and the breathalyzer are preferably physically uncoupled (e.g., in wireless communication) during broadcasting of the unique signature, such that Block S140 does not require physical coupling between the mobile computing device and the breathalyzer. However, the mobile computing device and the breathalyzer can alternatively be in any other suitable configuration in Blocks S140 and S150.

In relation to breath sample signal authentication, Blocks S140 and S150 are preferably performed according one or more embodiments, variations, and examples of sample authentication in a remote monitoring application, as described in U.S. application Ser. No. 14/602,919 entitled “Method and System for Remotely Monitoring Intoxication” and filed on 22 Jan. 2015, incorporated herein in its entirety by this reference. As such, in a specific example, Blocks S140 and S150 can include broadcasting a unique pattern of light emission from the breathalyzer and detecting the unique pattern of light emission using a camera module (e.g., video camera) of the mobile computing device, proximal in time to a time of breath sample provision by the individual. Sample authentication in Blocks S140 and S150 can, however, be performed in any other suitable manner.

In a first variation, Block S150 includes: using a sensor subsystem of the mobile computing device: authenticating the breath sample upon detection of the unique signature and 2) generating a location data entry of the user. In relation to this variation of Block S150, the location data entry can be generated from one or more of: a GPS subsystem, a triangulation system, and any other suitable system or apparatus that enables identification of current and/or past locations of the individual. The location determination systems can be incorporated with one or more of: an OBD adaptor of the OBD module of Block S110, the mobile computing device of the individual, a wearable computing device (e.g., wrist-borne wearable device, head-mounted wearable device, etc.), a subsystem or component of the computing system implemented in blocks of the method 100, and any other suitable system component associated with the method 100. Generating and providing location data in Block S150 can, however, be performed in any other suitable manner.

Block S150 can include Block S152, which recites: receiving the breath sample and the authentication signal, and Block S154, which recites: generating a verification assessment that validates provision of the breath sample by the user, based upon the breath sample signal and the authentication signal. Blocks S152 and S154 preferably occur at a remote server in communication with the mobile computing device, and together function to enable remote authentication of the breath sample provision by the user as described above and confirmation by a remote entity that the desired user provided an appropriate breath sample. Alternatively or additionally, Blocks S152 and S154 can be implemented at any suitable portion of the computing system or related elements, and/or performed independently at different portions of the computing system (e.g., the breath sample signal and the authentication signal are received at the remote server, and the verification assessment is generated at the mobile device in cooperation and communication with the remote server).

Block S160 recites: generating an intoxication assessment of the user, based on the breath sample signal, which functions to enable determination of the degree of intoxication of the user. Block S160, in variations, can include determining a value of an intoxication metric for the individual based upon the breath sample signal, which enables determination of the individual's level of intoxication in a quantitative manner. The intoxication metric is preferably a blood alcohol concentration (BAC), which can be determined from the breath sample signal generated as described in relation to Block S120 above; however, the intoxication metric can alternatively be any other suitable metric characterizing intoxication of the user. In variations, a BAC value corresponding to the breath sample can be determined based upon the magnitude of an electrical signal produced when alcohol in the user's breath reacts with a sensing element of the sensor (e.g., a current magnitude produced by a platinum-alcohol oxidation reaction for a fuel cell sensor, a change in electrical resistance produced by an alcohol-dioxide reaction for a semiconductor sensor, etc.). In other variations, a BAC value corresponding to any other suitable type of sample from the user can be determined based upon an electrical signal produced in response to irradiation of the sample (e.g., by way of an electrical pulse generated in response to absorption of infrared light by the sample, using a spectrophotometer), by way of a detected chemical change (e.g., as exhibited by a color change) in response to a chemical reaction between the sample and a chemical additive, or in any other suitable manner.

In Block S160, the value of the intoxication metric can be determined in-app at the native application executing at the mobile computing device of the individual. Additionally or alternatively, the value of the intoxication metric can be determined on-board using computing modules of the breathalyzer. Additionally or alternatively, the value of the intoxication metric can be determined in the cloud and/or at a remote server in communication with the mobile computing device, and the value of the intoxication metric can then be transmitted to and/or from other elements of the computing system. However, the value of the intoxication metric can be determined at any other suitable component of the system associated with the method 100.

In relation to Block S160, the breath sample signal is preferably received in real-time at computing system components configured to determine the value of the intoxication metric, such that determination of the intoxication metric is performed proximal in time to (e.g., immediately after) detection of a vehicle-user interaction and provision of the breath sample by the individual. Furthermore, authentication of the breath sample and/or the individual is also preferably performed in near real time and proximal in time to activation of the vehicle and breath sample provision by the individual. However, signal transmission, determination of the intoxication metric, and authenticating the breath sample/individual can additionally or alternatively be implemented in any other suitable manner.

Block S170 recites: performing an action configured to deter drunk driving by the individual, which functions to use the intoxication metric derived from Blocks S110-S160 to prevent potentially dangerous behavior by the individual. In Block S170, the action can be associated with an entity monitoring the individual (e.g., a guardian, a relative, a friend, a parole officer, acquaintance, employment-related entity, etc.) and/or the vehicle that the individual intends to use. In this and related examples, Block S170 can occur at a third time point proximal in time to a second time point (e.g., within 1 second, within 5 seconds, within 5 minutes, within 60 minutes, etc., relative to the second time point), wherein the user is prompted to provide a breath sample at the second time point. In order to trigger performance of the action, Block S170 can include Block S172, which recites: comparing the value of the intoxication metric to a threshold value. Performance of the action is preferably triggered if the value of the intoxication metric is greater than or equal to the threshold value; however, performance of the action can alternatively be triggered based upon any other suitable comparison. In specific examples, the threshold value can be a BAC value greater than 0.00 BAC, can be a value under 0.08 BAC, or can alternatively be any other suitable threshold value. The threshold value can be set by the entity monitoring the individual or can alternatively be set in any other suitable manner.

In one variation, the action performed in Block S170 can include transmitting a notification to a remote entity associated with the user, based on the intoxication assessment. The notification can be transmitted to one or more entities, and can function to alert the remote entity(s) based on the value of the intoxication assessment and/or intoxication metric and/or value. The remote entity can be the entity monitoring the individual, an entity socially associated with the individual (e.g., a friend on a social networking system), or be any other suitable entity. The notification can inform the entity of one or more of: the value of the intoxication metric, a location (e.g., a past location, a current location, etc.) of the individual, a time point at which the individual provided the breath sample, and analyses associated with authentication of the breath sample (e.g., a positive authentication result, a negative authentication result, an indeterminate authentication result, etc.), and any other suitable information. As such, the “threat” of notification of the entity monitoring the individual can serve as a deterrent in preventing drunk driving behavior by the individual.

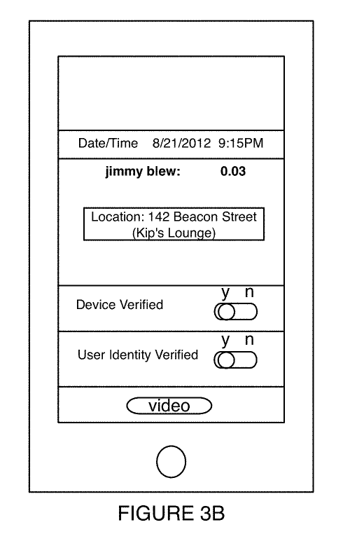

In related variations, the notification can inform the entity of the individual's current intoxication state, which in examples informs the entity that the user is: above a legal limit of intoxication, below a legal limit of intoxication, or at an unknown intoxication state relative to a legal limit of intoxication. In examples, generating the notification can include generating a rendering of an analysis, including one or more of: a graphical representation of the user's value of the intoxication metric relative to a legal limit, a trend in the value of the user's intoxication metric over time, and a graphical representation of assurance that the breath sample provided was not a fraudulent sample, examples of which are shown in FIGS. 3A-3B. In these examples and variations, the notification can be transmitted in any suitable manner (e.g., within a messaging client, within an application executing at a mobile computing device of the entity, by an online social network, in person, etc.).

Additionally or alternatively, in another variation, the action performed in Block S170 can include adjusting operation parameters of the vehicle to be driven by the individual. In examples, the action can include one or more of: an action that prevents the vehicle from starting (e.g., by disabling the ignition subsystem of the vehicle), an action that places a maximum speed limit at which the vehicle can be operated, an action that limits the distance over which the vehicle can be driven, an action that activates an automated (e.g., self-driving) function of the vehicle, an action that automatically drives the vehicle to a destination desired by the entity (e.g., a home of the entity or the individual), and any other suitable action. In one specific application, information (e.g., licensed information) including a database of codes specific to the vehicle(s) associated with the method 100 can be used to disable the vehicle ignition and/or modify functionality of any other vehicle subsystems through the OBD module. In these and related variations and applications, the vehicle operating parameter modification can be determined based on (e.g., calculated from, selected using, etc.) the value of the intoxication metric and/or intoxication assessment. The adjusted vehicle operation parameters (e.g., vehicle instructions) can be communicated to the vehicle: directly from the processing system generating the instructions, indirectly (e.g., through a proximal user device, through the diagnostic module, etc.), or otherwise sent to the vehicle.

Additionally or alternatively, in another variation, the action performed in Block S170 can include an action that promotes safety of the individual. For instance, the action performed in Block S170 can include initiating an alternative ride solution for the individual including one or more of setting up a ride service (e.g., Uber, Lyft) for the individual, calling a cab company to pick up the individual, and any other suitable ride solution for the individual. The action performed in Block S170 can additionally or alternatively include displaying a warning message at a display of the mobile computing device of the user, wherein the warning message includes a likelihood of harm to the user based on the value of the intoxication metric. As such, the user could be deterred from driving when provided with the knowledge that doing so comes with significantly increased likelihood of personal injury or injury to others (e.g., based on statistical analysis of traffic injuries and/or fatalities resulting from alcohol intoxication above a certain threshold).

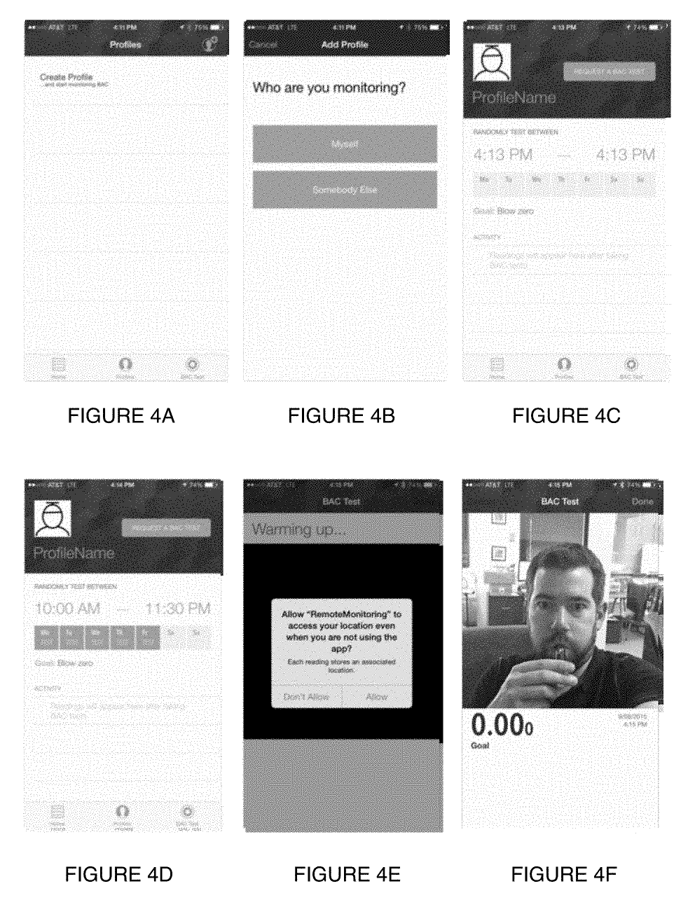

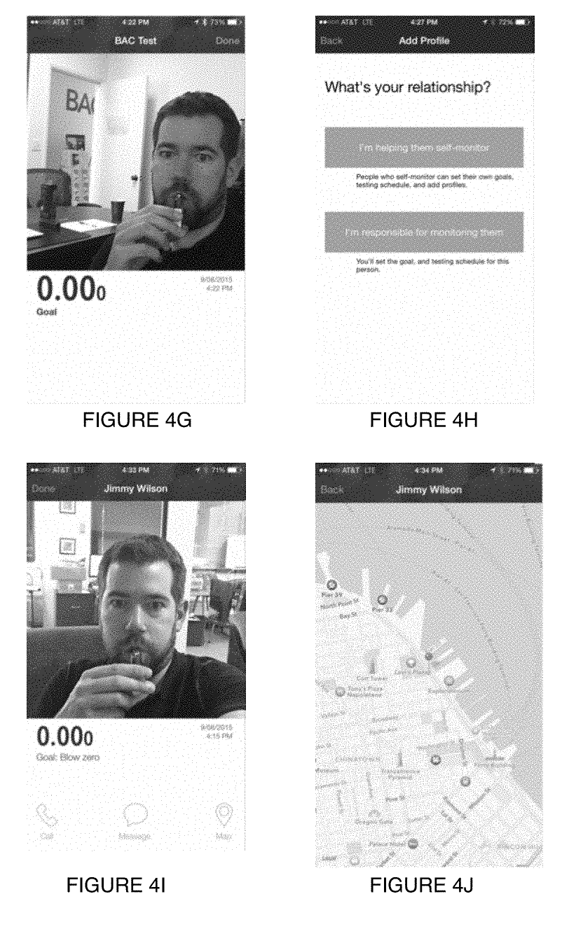

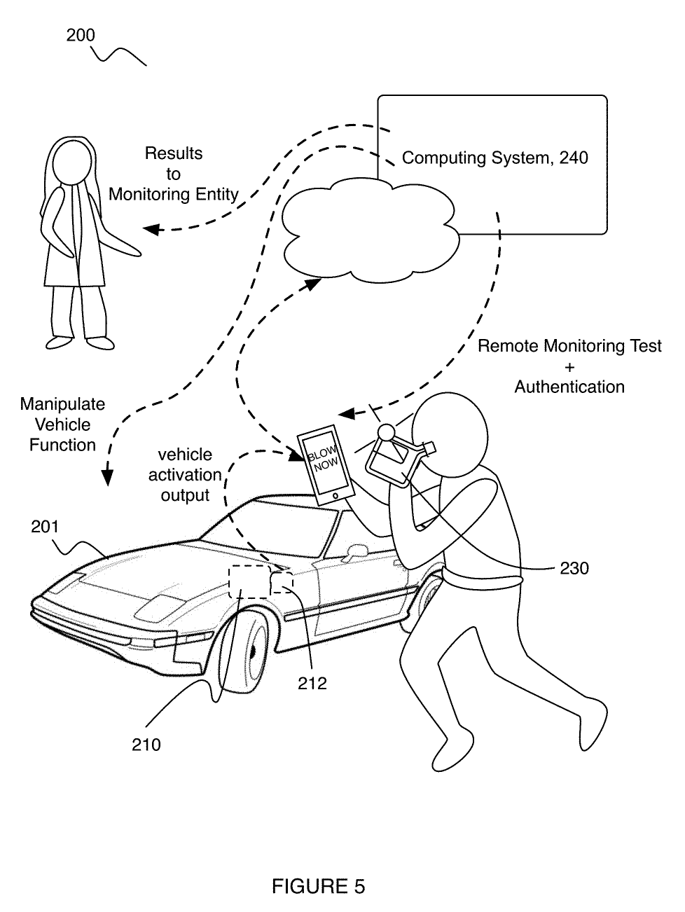

In a specific application of a portion of the method 100, as shown in FIGS. 4A-4J, a monitoring entity can be prompted, within an application, to create a profile for an individual to monitor (e.g., as in FIGS. 4A, 4B, and 4H); to provide monitoring parameters (e.g., testing times, randomness of testing, threshold BAC levels, etc.) associated with testing the individual (e.g., as in FIGS. 4C and 4D); to be able to manually request a test of the individual's impairment (e.g., as in FIGS. 4C and 4D); to render or report data associated with testing of the individual, including one or more of image/video data of the individual (e.g., as in FIGS. 4E, 4F, and 4G); to contact the individual (e.g., as in FIG. 4I); and to determine a location of the individual (e.g., as in FIGS. 4I and 4J). However, variations of the specific application can additionally or alternatively be implemented in any other suitable manner.

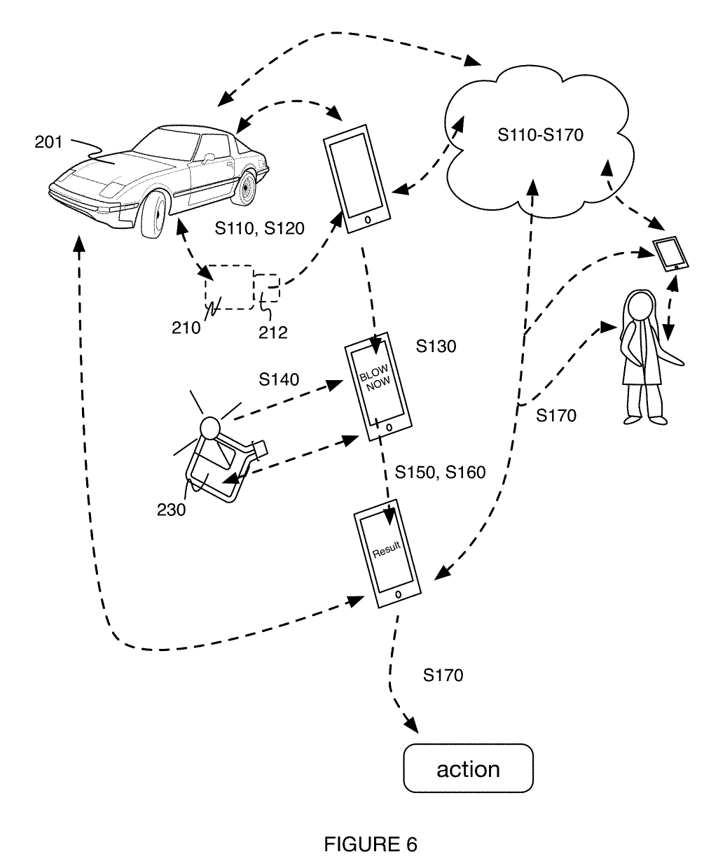

In other specific applications of a portion of the method 100, as shown in FIG. 6, data can flow through several pathways through various elements of the computing system. In a first example, a diagnostic module transmits an output, indicative of vehicle operation, to a mobile device, wherein the mobile device queries a cloud-based server to determine if the mobile device user is on a list of users that should provide a breath sample when interacting with a vehicle. The cloud-based server then transmits instructions to the mobile device to launch a native application, which prompts the user to provide a breath sample (and generates an authentication signal associated with the breath sample as described above, in relation to Blocks S140 and S150), and transmits the results to the cloud-based server for validation and/or verification (e.g., based on biometric analysis). The cloud-server then performs an action in response to the breath sample analysis, validation, and/or verification (e.g., notifying a remote entity that the user has provided a breath sample and/or that the provided breath sample has satisfied a condition).

In a second example, the output is received from the diagnostic module by the mobile device, which processes the output within a client application running on the mobile device to determine that a vehicle-user interaction has occurred. The client application further establishes the identity of the user and correlates that identity with a list of user identities, determining that the user should be prompted to provide a breath sample. The client application prompts the user to provide a breath sample, and receives the breath sample while also generating an authentication signal associated with receipt of the breath sample. The client application then transmits a breath sample signal based on the breath sample, as well as the authentication signal, to a cloud-based server, which then provides the authentication signal to a remote entity (e.g., at display of a mobile device of the remote entity) for verification assessment. The results of the verification assessment is then provided to the cloud-based server by the remote entity, wherein the cloud-based server then transmits instructions to perform an action to the mobile device of the user.

In a third related example, the vehicle (and/or a diagnostic module coupled to the vehicle) transmits an output indicative of a vehicle-user interaction directly to a cloud based server, before subsequent Blocks of the method 100 are performed in relation other portions of the computing system (e.g., a mobile device of the user). In other related applications and examples of the method 100, data (e.g., signals, inputs, outputs, analyses, etc.) can be transferred between any suitable elements of the computing system in any suitable order.

In a fourth example, the diagnostic module transmits a signal indicative of vehicle-user interaction (e.g., the output or a different signal) to a third-party remote computing system (e.g., directly or through the mobile device), the third-party remote computing system sends the output to the remote computing system in response to signal receipt, the remote computing system determines whether a testing condition has been met, and in response to determination that the testing condition has been met, instructs the mobile device (e.g., the client on the user device) and/or breathalyzer to initiate testing (e.g., directly or through one or more intermediary communication systems, such as the vehicle communication system).

The method 100 can, however, include any other suitable blocks or steps configured to facilitate remote monitoring of a user, in relation to states of impairment or intoxication, and preventing dangerous behaviors from being performed by the individual. For instance, the method 100 can include blocks configured to facilitate transmission of incentives (e.g., monetary incentives, motivational incentives, etc.) to individual whenever the individual behaves in a positive manner (e.g., does not drive attempt to drive while intoxicated, etc.). Additionally or alternatively, applications of the method 100 can be expanded to facilitate remote monitoring of any other suitable type of impairment (e.g., marijuana-induced impairment, other substance-induced impairment, etc.), and preventing the individual from performing unsafe behaviors in associated states of impairment. Furthermore, as a person skilled in the art will recognize from the previous detailed description and from the figures and claims, modifications and changes can be made to the method 100 without departing from the scope of the method 100.

### 2. System

As shown in FIG. 4, an embodiment of a system 200 preventing drunk driving by an individual includes an OBD module 210 including an OBD adaptor 212 coupled to a vehicle 201 intended to be driven by the individual; a mobile computing device 220 of the individual, the mobile computing device in communication with the OBD adaptor 212 and configured to 1) receive an output indicative of a vehicle-user interaction from the OBD adaptor and 2) prompt the individual to provide a breath sample upon reception of the output; a breath sample acquisition device 230 in communication with the mobile computing device and configured to generate a breath sample signal upon reception of the breath sample and facilitate generation of an authentication of the breath sample from the user; and a computing system 240 in communication with the mobile computing device 220 that 1) generates a value of an intoxication metric from the breath sample signal and 2) based upon the value of the intoxication metric and the authentication, initiates performance of an action configured to prevent drunk driving by the individual.

The system 200 functions to provide a tool that prevents an individual from performing activities that could be life-threatening while in a state of inebriation. As such, the system 200 includes elements that cooperate to detect when the individual may be in a compromised state, intends to perform a dangerous activity, and provides a deterrent against the individual's performance of the activity. Furthermore, elements of the system are configured to prevent fraudulent sample submission by individuals attempting to avoid consequences of an intoxication level above a certain limit (e.g., a legal limit). The system 200 preferably enables an entity who is remote from the individual to monitor the user's intoxication levels, such that the user does not need to be in the physical presence of the entity monitoring his/her intoxication. In examples, the entity can be a guardian, a supervisor (e.g., work supervisor, probation officer, etc.), a caretaker, a family member, an acquaintance, an employment-related entity (e.g., coworker, boss, human resources entity, employer, etc.) or any other suitable entity associated with the user who has an interest in monitoring the user's intoxication. In variations, the system 200 can be configured to perform at least a portion of the method 100 described in Section 1 above, and can additionally or alternatively be configured to perform any suitable method that facilitates remote monitoring of a user's intoxication.

The system 200 can include elements (e.g., OBD module elements) as described in Section 1 above. The system 200 can additionally or alternatively include one or more embodiments, variations, and examples of system elements (e.g., breath sample acquisition device components, mobile computing device components, computing system components, etc.) described in U.S. application Ser. No. 14/169,029 entitled “Method and System for Monitoring Intoxication” and filed on 30 Jan. 2014, U.S. application Ser. No. 14/602,909 entitled “Method and System for Remotely Monitoring Intoxication” and filed on 22 Jan. 2015, and U.S. application Ser. No. 14/631,125 entitled “Method and System for Monitoring Intoxication” and filed on 25 Feb. 2015, each of which is incorporated herein in its entirety by this reference. Variations of the system 100 can, however, be implemented at least in part using any other suitable system elements.

Variations of the method 100 and system 200 include any combination or permutation of the described components and processes. Specific examples of the method 100 and system 200 are illustrative and not prohibitive. Furthermore, various processes of the preferred method can be embodied and/or implemented at least in part as a machine configured to receive a computer-readable medium storing computer-readable instructions. The instructions are preferably executed by computer-executable components preferably integrated with a system and one or more portions of the control module 155 and/or a processor. The computer-readable medium can be implemented in the cloud and/or stored on any suitable computer readable media such as RAMs, ROMs, flash memory, EEPROMs, optical devices (CD or DVD), hard drives, floppy drives, or any suitable device. The computer-executable component is preferably a general or application specific processor, but any suitable dedicated hardware device or hardware/firmware combination device can additionally or alternatively execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Although omitted for conciseness, the preferred embodiments include every combination and permutation of the various system components and the various method processes, wherein the method processes can be performed in any suitable order, sequentially or concurrently.

As a person skilled in the art will recognize from the previous detailed description and from the figures and claims, modifications and changes can be made to the preferred embodiments of the invention without departing from the scope of this invention defined in the following claims.

We claim:

1. A method for remotely monitoring intoxication of a user, comprising:
receiving an output, indicative of a vehicle-user interaction, from a diagnostic module electrically coupled to a vehicle;
determining a user characteristic of the user in response to receiving the output;
in response to determining the user characteristic, determining that the user characteristic satisfies a testing condition;
in response to determination that the user characteristic satisfies the testing condition, prompting the user, at an application executing on a mobile computing device associated with the user, to provide a breath sample;
at a breath sample acquisition device in communication with the mobile computing device:
receiving a breath sample from the user, and
transmitting a breath sample signal derived from the breath sample to the mobile computing device;
at a processing system in communication with the mobile computing device, receiving the breath sample signal;
generating an intoxication assessment of the user, based on the breath sample signal; and
transmitting a notification to a remote entity associated with the user, based on the intoxication assessment.
